(19) United States
(12) Patent Application Publication
(10) Pub. No.: US 2004/0133057 A1
(43) Pub. Date: Jul. 8, 2004

(54) Gaseous hydrocarbon-oxygen bubble tank mixer

(75) Inventors: Yi Jiang, Ponca City, OK (US); Harold A. Wright, Ponca City, OK (US); Doug S. Jack, Ponca City, OK (US)

Correspondence Address: DAVID W. WESTPHAL, CONOCOPHILLIPS COMPANY, P.O. BOX 1267, PONCA CITY, OK, 74602-1267, US

(73) Assignee: ConocoPhillips Company, Houston, TX

(21) Appl. No.: 10/732,877

(22) Filed: Dec. 10, 2003

Related U.S. Application Data
(60) Provisional application No. 60/437,685, filed on Jan. 2, 2003.

(51) Int. Cl. C07C 1/00; B01J 10/00

(52) U.S. Cl. 585/733; 422/189; 422/190; 422/224; 422/225; 422/198; 422/228; 585/638

(57) ABSTRACT

The invention relates to methods and apparatus for mixing a plurality of gases. The preferred embodiments of the invention comprise forming bubbles of at least two gases injected separately into a liquid, and passing said bubbles through a gas-induced turbulent liquid region to enhance gas transfer between bubbles and to thereby mix the at least two gases. Creating the gas-induced turbulent liquid region preferably includes using a high gas superficial velocity, and may further include using powered mechanical devices, static internal structures, fluid recirculation, or combinations thereof. The gas mixture is preferably supplied to a reaction zone. In one embodiment a bubble tank mixer supplies a gas mixture comprising oxygen and a hydrocarbon gas to an oxidation reaction zone disposed above said mixer. In alternative embodiments the reaction zone and mixer may be integrated into the same vessel.

### CROSS-REFERENCE TO RELATED APPLICATIONS

The present application claims the benefit of 35 U.S.C. 111(b) Provisional Application Serial No. 60/437,685 filed Jan. 2, 2003 and entitled “Gaseous Hydrocarbon-Oxygen Bubble Column Mixer,” which is hereby incorporated by reference herein for all purposes.

### STATEMENT REGARDING FEDERALLY SPONSORED RESEARCH OR DEVELOPMENT

Not applicable

### TECHNICAL FIELD OF THE INVENTION

The present invention relates generally to methods and apparatus for mixing gases. More specifically, the present invention relates to methods and apparatus for mixing a feed gas to supply to an oxidation reaction zone.

### BACKGROUND OF THE INVENTION

Natural gas, found in deposits in the earth, is an abundant energy resource. For example, natural gas commonly serves as a fuel for heating, cooking, and power generation, among other things. The process of obtaining natural gas from an earth formation typically includes drilling a well into the formation. Wells that provide natural gas are often remote from locations with a demand for the consumption of the natural gas.

Thus, natural gas is conventionally transported large distances from the wellhead to commercial destinations in pipelines. This transportation presents technological challenges due in part to the large volume occupied by a gas. Because the volume of an amount of gas is so much greater than the volume of the same number of gas molecules in a liquefied state, the process of transporting natural gas typically includes chilling and/or pressurizing the natural gas in order to liquefy it. However, this contributes to the final cost of the natural gas and may not be economical for formations containing small amounts of natural gas.

Formations that include small amounts of natural gas may include primarily oil, with the natural gas being a byproduct of oil production that is thus termed associated gas. In the past, associated gas has typically been flared, i.e., burned in the ambient air. However, current environmental concerns and regulations discourage or prohibit this practice.

Further, naturally occurring sources of crude oil used for liquid fuels such as gasoline, jet fuel, kerosene, and diesel fuel have been decreasing and supplies are not expected to meet demand in the coming years. Fuels that are liquid under standard atmospheric conditions have the advantage that in addition to their value, they can be transported more easily in a pipeline than natural gas, since they do not require liquefaction.

Thus, for all of the above-described reasons, there has been interest in developing technologies for converting natural gas to more readily transportable liquid fuels, i.e. to fuels that are liquid at standard temperatures and pressures. One method for converting natural gas to liquid fuels involves two sequential chemical transformations. In the first transformation, natural gas or methane, the major chemical component of natural gas, is reacted with oxygen to form syngas, which is a combination of carbon monoxide gas and hydrogen gas. In the second transformation, known as the Fischer-Tropsch process, carbon monoxide is reacted with hydrogen to form organic molecules containing carbon and hydrogen.

Catalytic partial oxidation is one process used to form syngas by attempting to perform all of the partial oxidation reactions on a highly active catalyst so as to convert the hydrocarbon catalytically at a high rate. For example, the contact times involved in a typical catalytic partial oxidation reaction may be on the order of milliseconds. Thus, for catalytic partial oxidation, it is often preferable to premix a hydrocarbon-containing feed, such as methane or natural gas, with a molecular oxygen-containing feed at sufficient temperature, pressure and velocity in order to enable the catalytic reaction to proceed at the short contact times required so that the chemistry occurs at the correct stoichiometry throughout the catalytic zone.

Therefore, an often desired component of a commercial scale operation is an apparatus to premix the hydrocarbon-containing gas, such as methane or natural gas, and the molecular oxygen-containing gas, such as air or substantially pure O₂, at the desired temperature, pressure, and flow rate. The same feed conditions that are generally conducive to efficient operation of the partial oxidation process, however, could lead to reactions that are less desirable, and possibly even hazardous, such as the ignition and the detonation of reactant gases.

As described in U.S. Pat. No. 6,267,912, which is incorporated herein by reference, catalytic partial oxidation processes attempt to eliminate gas phase oxidation reactions entirely, so that all of the partial oxidation reactions take place on the catalyst surface. The reactants are contacted with the catalyst at a very high space velocity, so that gas phase reactions are minimized. Gas phase reactions are undesirable because they can increase the occurrence of undesired combustion reactions (producing steam and carbon dioxide) that lead to hotspots, damage the catalyst, and accelerate its deactivation.

FIG. 1 is a schematic representation of a prior art partial oxidation system 100 having a partial oxidation reactor 110 and a reactant gas mixer 120. A hydrocarbon stream 130 and oxygen stream 140 feed into reactant gas mixer 120. Because it is often desired that the reactant gases have an elevated temperature when entering reactor 110, the gases that enter mixer 120 are often preheated, either before or during mixing, to the desired temperature for reaction.

One problem with such mixing processes is that heated mixtures of oxygen and hydrocarbons, at pressures of interest for syngas production, are highly reactive and can be explosive. Thus, it is often preferred to utilize techniques that increase the controllability of the process in order to avoid pre-ignition and pre-reaction of the gases. One technique used in mixing the reactants is to place the mixing nozzles very close to the reaction zone such that there is a very short time between the reactants being mixed and contacting the catalyst. This technique often involves placing the mixing apparatus in close proximity to the reactor, which may make maintenance of the mixing apparatus difficult and requires that the mixer be designed to withstand the extreme environment of the partial oxidation reactor.

As described in U.S. Pat. No. 4,269,791, which is incorporated herein by reference, the hydrogen and oxygen are mixed at a ratio of 95:5 H2-O2 through turbulent liquid media for diving applications where the mixing primarily occurs through gas diffusion in liquid phase, not through gas bubble interactions by coalescence and breakage. The bubble sizes described in U.S. Pat. No. 4,269,791 are very small [in the range of 50 to 100 micron (μm)], and do not induce massive liquid turbulence. Therefore, U.S. Pat. No. 4,269,791 discloses the use of a pump to recycle the liquid to generate liquid turbulence.

Thus, there remains a need in the art for methods and apparatus to improve the mixing of gases in a safe and efficient manner in view of feeding the gas mixture to a reaction zone, particularly to improve the mixing of hydrocarbon gas (such as methane, ethane, and/or natural gas) and oxygen to feed an oxidation process, specifically a partial oxidation process. Therefore, the embodiments of the present invention are directed to methods and apparatus for mixing gases that seek to overcome these and other limitations of the prior art.

### SUMMARY OF THE PREFERRED EMBODIMENTS

Accordingly, there are provided herein methods and apparatus for mixing a plurality of gases, preferably a hydrocarbon and an oxidant. The basis of the invention is to apply energy to a liquid to create a turbulent liquid flow and using this turbulent liquid flow to mix a plurality of gases passing through said turbulent liquid. The turbulence imparted to the liquid is preferably achieved by passing gases at a high gas superficial velocity so as to create a gas-induced liquid turbulence, and the intensity of liquid turbulence may be supplemented by using other means such as employing one or more mechanical devices. The energy imparted to the liquid, through which gas flows, provides the energy necessary for promoting gas bubble collisions, for overcoming the gas bubble surface tension so as to enhance bubble coalescence, and for breaking up gas bubbles into smaller sizes. The preferred embodiments of the present invention are characterized by a mixing apparatus that utilizes a gas-induced liquid turbulent region within the liquid to mix multiple streams of gas as they are injected and dispersed into bubbles as they pass through the liquid. As the gas bubbles move through the turbulent liquid, the bubbles repeatedly collide, coalesce, and break-up, providing a well mixed gas suitable for use as a reactant gas in a reactor. Means for creating the gas-induced turbulent liquid region preferably includes using a high gas superficial velocity, and may be further supplemented by using powered mechanical devices, static internal structures, fluid recirculation, or combinations thereof.

One embodiment of a reactor system utilizing such bubble mixer for forming a reactant mixture comprising a hydrocarbon gas and an oxygen-containing gas includes a tank comprising a bottom half and containing a liquid; one hydrocarbon gas inlet located in the bottom half of the tank, wherein the hydrocarbon gas inlet comprises means of dispersing a hydrocarbon-containing gas into bubbles within said liquid; one oxidant gas inlet located near or at the bottom of the tank, wherein the oxidant gas inlet comprises means of dispersing an oxygen-containing gas into bubbles within said liquid; means of forming a gas-induced liquid turbulent region in at least a portion of said liquid sufficient to mix said bubbles of oxygen-containing gas and hydrocarbon-containing gas to provide a reactant gas; and a reactor body in fluid contact with said tank adapted to receive the reactant gas at conditions favorable for the production of reaction products.

The mixing tank preferably has a height-to-diameter aspect ratio between 1 and 15. Gaseous streams of hydrocarbon and oxygen are injected into the bottom half of the mixing tank through separate gas distribution systems at a superficial velocity which can induce sufficient liquid turbulent flow. As the separate hydrocarbon and oxygen bubbles rise through the tank, turbulence within the liquid causes the bubbles to collide, coalesce, and break-up, thereby mixing the gases contained in the separate bubbles. The mixed reactant gas can then be collected from the top of the mixing tank and is suitable for use in a reactor.

In alternative embodiments, the tank may be equipped with a powered mechanical device, a fluid circulation system, a static internal structure, or combinations thereof for further enhancing turbulence intensity within the liquid. The powered mechanical device may comprise at least one paddle, at least one stirrer, at least one impeller, at least one propeller, or combinations thereof. The static internal structure may comprise at least one baffle, at least one perforated plate, a packing material, a heat-exchange device, or combinations thereof. The fluid circulation system preferably comprises a reactant gas recycling loop with a compressor.

The tank may also include heat exchange tubes, or other means for providing a control of liquid temperature in order to preheat the reactant gas to control liquid vaporization for use in a reactor, and/or to vary the solubility of components in the feed gases into the liquid.

As the hydrocarbon and oxygen bubbles collide, some bubbles may be formed that contain an oxygen-rich gas mixture comprising a hydrocarbon that, under certain conditions, may be subject to explosion. One advantage of the reactor system employing such bubble tank mixer is that the turbulence within the liquid is expected to limit the size of bubbles that may be formed, thus limiting the volume of gas in any single bubble. Thus, the volume of gas subject to explosion would be relatively small. This small volume would also effectively be isolated from other gas volumes by the liquid within the tank. Therefore, it is desired that the liquid within the tank be suited to prevent the propagation of any local explosion beyond the gas bubble immediately involved.

The intensity of liquid turbulence in the gas-induced liquid turbulent region created in the bubble mixer tank can be controlled by the total superficial velocity (U_{G}) of gaseous streams entering the tank, and optionally increased by additional energy input from for example the rotating speed of the mechanical agitator.

The invention also relates to a method for forming a reactant gas mixture in a safe and efficient manner before being reacted, said method comprising the steps of: providing a tank containing a liquid; injecting a first feed gas into said liquid in a manner effective to subdivide the first feed gas into bubbles within the liquid; separately injecting a second feed gas into said liquid in a manner effective to subdivide the second feed gas into bubbles within the liquid; forming a gas-induced liquid turbulent region in at least a portion of said liquid; passing bubbles of said first and second feed gases through said gas-induced liquid turbulent region so as to induce gas transfer between the bubbles and to form a reactant gas mixture comprising the first and second feed gases; and supplying at least a portion of the reactant gas mixture to a reaction zone.

In one embodiment of an oxidation system, a catalytic partial oxidation reactor is supplied with reactant gas prepared in a bubble tank mixer. The catalytic partial oxidation reactor is preferably disposed above the mixer, which comprises a mixing tank filled with a liquid. In some embodiments, the reactor and mixer may be integrated into the same vessel. The components of the reactant gas, namely a hydrocarbon, such as one or more gaseous hydrocarbons like methane or natural gas, and an oxygen containing gas, are injected preferably into the bottom half of the mixing tank. The liquid is maintained at a sufficient turbulence such that, as the bubbles of hydrocarbon and oxygen rise through the fluid, they collide, coalesce, and break-up at a high frequency. The highly-frequent bubble interactions provide for a thoroughly mixed reactant gas exiting the mixing tank. The induced liquid turbulence can be controlled by gas superficial velocity and optionally by mechanical agitation speed or the use of liquid flow-hindering devices.

In one embodiment relating to a process for the oxidation of hydrocarbon, the process includes forming a reactant gas mixture comprising a hydrocarbon gas and an oxygen-containing gas in a bubble tank mixer; supplying at least a portion of the reactant gas mixture to a reactor, and reacting at least a portion of said hydrocarbon gas with oxygen to form a reaction product. The oxidation reaction preferably includes a partial oxidation reaction, and the hydrocarbon-containing gas contains mainly methane, such that the reaction product comprises a mixture of hydrogen and carbon monoxide (syngas).

Additionally, the invention further involves the production of C₅₊ hydrocarbons from a hydrocarbon-containing gas. The process comprises forming a hydrocarbon/oxygen mixture in a bubble mixer tank; forming a syngas stream by passing the hydrocarbon/oxygen mixture through an oxidation reaction zone; feeding at least a portion of the syngas stream to a hydrocarbon synthesis reactor comprising a hydrocarbon synthesis catalyst; and converting at least a portion of said syngas stream in the hydrocarbon synthesis reactor to form C₅₊ hydrocarbons: The C₅₊ hydrocarbons comprise hydrocarbons with 5 or more carbon atoms.

Thus, the present invention comprises a combination of features and advantages that enable it to substantially increase the efficiency and safety of mixing hydrocarbons and an oxygen to feed a catalytic partial oxidation process. These and various other characteristics and advantages of the present invention will be readily apparent to those skilled in the art upon reading the following detailed description of the preferred embodiments of the invention and by referring to the accompanying drawings.

### BRIEF DESCRIPTION OF THE DRAWINGS

For a more detailed understanding of the preferred embodiments, reference is made to the accompanying Figures, wherein:

FIG. 1 is a schematic view of a prior art partial oxidation system;

FIG. 2 is a schematic view of one embodiment of an oxidation system including a mixing tank;

FIG. 3 is a schematic view of one embodiment of a mixing tank;

FIGS. 4A-4D illustrate variations of bubble coalescence;

FIGS. 5A-5C illustrate variations of bubble break-up;

FIG. 6 is a schematic view of one embodiment of a mixing tank including a mechanical stirrer;

FIG. 7 is a schematic view of one embodiment of a mixing tank including partition plates;

FIG. 8 is a schematic view of one embodiment of a mixing tank with a gas circulation system;

FIG. 9 is a schematic view of one embodiment of a mixing tank including heating tubes;

FIG. 10 is a schematic view of an alternative embodiment of an oxidation system; and

FIG. 11 is a schematic view of one embodiment of a mixing tank including packing material.

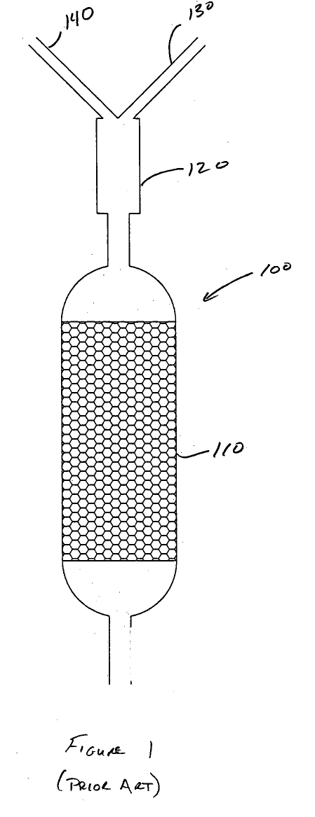

### DETAILED DESCRIPTION OF THE PREFERRED EMBODIMENTS

In the description that follows, like parts are marked throughout the specification and drawings with the same reference numerals, respectively. The drawing figures are not necessarily to scale. Certain features of the invention may be shown exaggerated in scale or in somewhat schematic form and some details of conventional elements may not be shown in the interest of clarity and conciseness.

The present invention relates to methods and apparatus for mixing at least two feed gases natural gas and oxygen to supply a reactant gas to an oxidation reaction. The preferred embodiments include mixing a hydrocarbon gas and an oxygen-containing gas to form a reactant gas mixture to be supplied to a partial oxidation zone. The hydrocarbon gas may comprise one or more hydrocarbons, such as methane, ethane, natural gas, or mixtures thereof. The present invention is susceptible to embodiments of different forms. There are shown in the drawings, and herein will be described in detail, specific embodiments of the present invention with the understanding that the present disclosure is to be considered an exemplification of the principles of the invention, and is not intended to limit the invention to that illustrated and described herein.

In particular, various embodiments of the present invention provide a number of different methods and apparatus for mixing gases. Reference is made to mixing a hydrocarbon gas and oxygen for an oxidation reaction, such as non-catalytic and catalytic partial oxidation reactions, but the use of the concepts of the present invention is not limited to mixing solely hydrocarbon gas and oxygen, or for use solely with an oxidation process, and can be used in any other mixing application. It is to be fully recognized that the different teachings of the embodiments discussed below may be employed separately or in any suitable combination to produce desired results.

FIG. 2 illustrates one embodiment of a partial oxidation system 200 having an oxidation reactor 210 supplied by a gases fed through a bubble tank mixer 220. Bubble tank mixer 220 includes mixing tank 230 that is filled with a suitable liquid. Mixing tank 230 preferably comprises a column, which has a height-to-diameter aspect ratio between 1 and 15. Gaseous hydrocarbon stream 240 and oxidant stream 250 provide feed gases that are injected separately into the bottom half of tank 230 through distribution systems 260 and 270, respectively. The bottom half of said tank corresponds to a region of the tank between 0 and H/2 where H is the total height of the tank and corresponds to the top of the tank. The injection of the feed gases 240 and 250 form bubbles 280 that move upward through the liquid in tank 230. Gas outlet 225 is disposed at the top of mixing tank 230 and provides a conduit to oxidation reactor 210. A recycle line 235 may be provided to recycle a portion of the reactant gas from outlet 225 back into mixing tank 230. Recycle line 225 should comprise a compressor (not illustrated) in order to delivery the portion of the reactant gas at a pressure suitable for reentry into mixing tank 230.

Oxidation reactor 210 may be any type of reactor that processes a feed gas containing at least one hydrocarbon and an oxidant. One preferred oxidation reactor comprises a catalytic partial oxidation reaction that reacts a hydrocarbon, such as methane, mixture of C1-C4 hydrocarbons or natural gas, with an oxygen containing gas to form a syngas product comprising a combination of hydrogen and carbon monoxide. Oxidation reactor 210 as illustrated in FIG. 2 comprises a catalyst structure; however it is not necessary that oxidation reactor 210 contains a catalyst structure, as a non-catalytic partial oxidation reaction is a suitable alternate oxidation reaction to which this invention could be applied. In some embodiments, the process comprises mixing a hydrocarbon-containing feedstock and an O₂-containing feedstock together in an O₂-to-carbon molar ratio of about 0.1:1 to about 0.8:1, preferably about 0.45:1 to about 0.65:1. Preferably the hydrocarbon-containing feedstock is at least 80% methane, more preferably at least 90%.

Pressure, residence time, amount of feed preheat and feed compositions also affect the reaction products. In a catalytic partial oxidation system, the preferred mixing systems supply a reactant gas mixture over, or through, the porous structure of the catalyst system in reactor 210 at a gas hourly space velocity of about 20,000-100,000,000 h⁻¹, preferably about 100,000-25,000,000 h⁻¹. In certain embodiments of the catalytic partial oxidation process, the temperature of the reactant gas mixture is preferably about 20° C.-750° C. In some embodiments, the catalytic partial oxidation process includes maintaining the reactant gas mixture at a pressure of about 100-32,000 kPa (about 1-320 atmospheres), preferably about 200-10,000 kPa (about 2-100 atmospheres), while contacting the catalyst. In these embodiments, it is therefore desirable that mixing tank 230 provide a flow of reactant gas that is preferably between 45 psig (300 kPa) and 500 psig (3350 kPa), at between −20° F. (−29° C.) and 1000° F. (538° C.), contains 0 to 100% O₂, and exits mixing tank 230 at between about 0.1 ft/s (0.03 m/s) and about 200 ft/s (61 m/s). The increased pressure in mixing tank 230 is expected to decrease oxygen solubility in the liquid, to minimize the need or size of a compressor between mixing tank 230 and reactor 210, and to increase the boiling point of the liquid so as to minimize the loss of liquid in the reactant gas mixture.

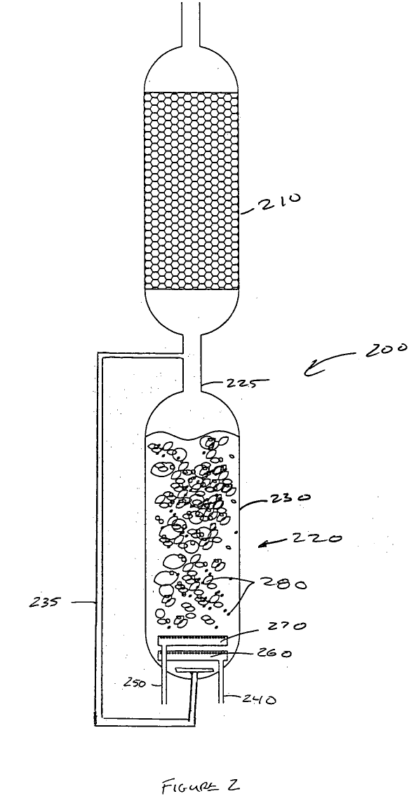

FIG. 3 shows one embodiment of mixing tank 230 filled with a liquid. A hydrocarbon gas, supplied by stream 240, is injected into tank 230 through distribution system 260. An oxygen-containing gas, supplied by stream 250, is injected into tank 220 through distribution system 270. Distribution systems 260 and 270 may be any arrangement of sparging rings, nozzles, inlets, or any combination thereof as may be useful to inject the feed gases in a desired concentration and distribution. As the hydrocarbon and oxidant gas bubbles travel through the liquid, turbulence within the liquid volume causes the bubbles to collide with each other. These collisions cause the bubble to repeatedly coalesce into larger bubbles and then break-up into smaller bubbles. It is this cycle of coalescing and breaking-up that mixes the hydrocarbon and oxidant gas such that, by the time the gas bubbles reach the top of tank 230, the gas is fully mixed in the desired ratio. A desirable residence time for the gas bubbles in the liquid phase is greater than 1 second, preferably between 2 seconds and 20 minutes. Since there is sufficient collision/coalescence/breaking events of the bubbles to cause a change in bubble size over the length of the gas-expanded bed, the type of gas distribution system 260 and 270 employed in delivering the feed gases is not critical.

The preferred liquid in mixing tank 230 may be any suitable liquid based on interaction with the feed gases and the temperature conditions sought to be maintained in mixing tank 230. The liquid may comprise water, an organic liquid, or a combination thereof. The organic liquid may comprise a hydrocarbon mixture such as hydrocarbon wax, lubricating oil, middle distillate including diesel, naphtha, gasoline, or mixtures thereof, whether the hydrocarbon mixture is obtained from processing of crude oil, tar sand, shale oil, or synthesized from synthesis gas by a hydrocarbon synthesis process such as utilizing Fischer-Tropsch reaction. Preferably the organic liquid comprises a synthetic hydrocarbon mixture such as diesel, naphtha, lubricating oil stock, and/or wax derived from a Fischer-Tropsch synthesis. A highly paraffinic mixture is quite desirable for the organic liquid. Additional suitable organic liquid may comprise a biofuel derived from biomass (such as biodiesel), alcohol, mineral oil, and/or one or more vegetable oils (canola oil, corn oil, soybean oil, and the like). For example, in low temperature applications water may be acceptable, while in higher temperature applications, a liquid with a higher boiling point may be desired. If the mixing is done at a temperature below 100° C., then water is the preferred liquid in mixing tank 230. If the mixing is done at a temperature greater than 100° C., then an organic liquid such as Fischer-Tropsch derived wax may be used.

A separation system such as a cooling unit, a condenser, a de-mister, or a combination thereof may be desirable to recover a portion of the carried-over liquid in the reactant gas upon exiting tank 230. Preferably, at least a portion of the recovered portion of the carried-over liquid is recycled to mixer tank 230; however it is not necessary to recycle a portion or all of the recovered liquid from reactant gas mixture. A make-up liquid stream (not illustrated) could be added to mixer tank 230 so as to maintain the height of the gas-expanded bed, and/or to maintain liquid inventory into the tank 230.

In some embodiments, the liquid may be reactive with at least one feed gas, however the liquid is preferably unreactive with the plurality of gases. The liquid is also preferably capable of containing any localized explosion within a particular gas bubble. It is also preferable to minimize the amount of gases absorbed by the liquid. The liquid in mixing tank 230 may initially absorb a component of a feed gas until saturation of this component is reached into the liquid. After saturation is reached, it is expected that there should not be significant additional absorption of this gaseous component from the feed gas. Although it is expected that gas-to-liquid-to-gas mass transfer does take place, however the operating conditions of the mixer tank should be designed to promote gas-to-gas mass transfer through bubble coalescences and breakages. In addition, the elevated pressure (greater than 300 kPa or 45 psig) in mixing tank 230 is expected to also minimize the solubility of components such as oxygen and hydrocarbons in liquids such as water or hydrocarbon mixture.

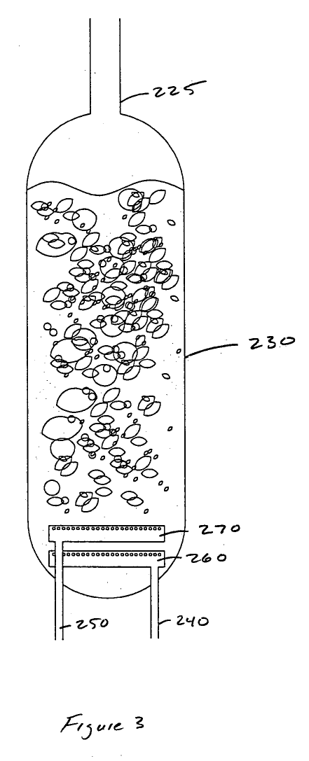

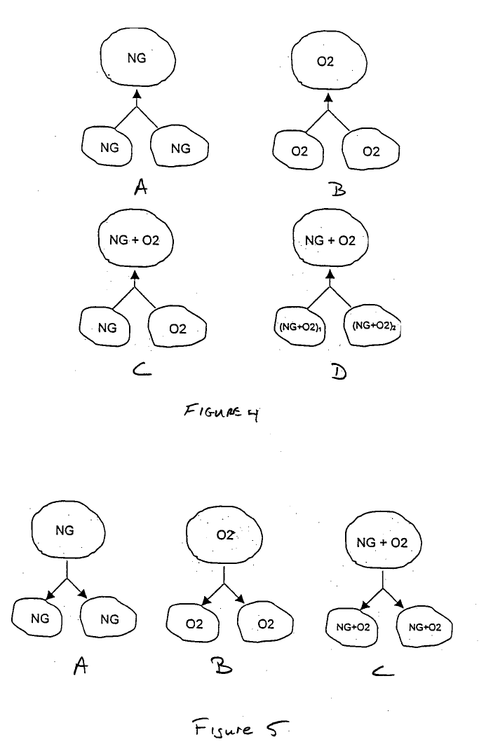

FIGS. 4A-4D illustrate the coalescing of gas bubbles from smaller bubbles into larger bubbles. In the figures, NG symbolizes natural gas and O₂ symbolizes oxygen. Even though natural gas is illustrated in FIGS. 4A-4D, it should be understood that the illustration is also suitable for any hydrocarbon-containing gas. FIG. 4A shows that when two small bubbles of natural gas collide and coalesce, a larger bubble of natural gas is formed. Correspondingly, as seen in FIG. 4B, when two small bubbles of oxygen collide and coalesce, a larger bubble of oxygen is formed. As depicted in FIG. 4C, when a bubble of oxygen collides and coalesces with a bubble of natural gas a larger bubble containing a mixture of natural gas and oxygen is formed. FIG. 4D illustrates that when small bubbles of mixed natural gas and oxygen collide and coalesce, the larger bubble will also contain a mixture of the two gases.

FIGS. 5A-5C illustrate the break-up of larger gas bubbles into smaller gas bubbles. FIGS. 5A and 5B illustrate that when a larger bubble containing only one gas divides into smaller bubbles, those smaller bubbles will also only contain that one gas. FIG. 5C shows that when a larger bubble, containing a mixture of gases in a certain concentration, breaks-up, the resultant smaller bubbles will contain substantially the same gas in substantially the same concentration as the larger bubble.

The repeated coalescence and break-up of bubbles is the mechanism relied on for mixing the feed gases into the desired reactant gas. Thus, the frequency with which the individual bubbles collide, coalesce, and break-up is critical to the preparation of the desired reactant gas. The frequency of collision, coalescence, and break-up is partially determined by the turbulence created within at least a region of the liquid volume. Turbulence in at least a region of the liquid is caused by the combined gas flow of the plurality of gases, preferably corresponding to a total gas superficial velocity between about 5 and 60 cm/sec, more preferably 10-60 cm/sec, more preferably 10-45 and still more preferably about 20-40 cm/sec. The total gas superficial velocities inside the mixing tank may be different than the velocity of the reactant gas mixture entering the reaction zone because of possible path restrictions (as illustrated in FIG. 2) or expansions (not shown) between the mixing tank and the reaction zone, which may accelerate or decelerate the reactant gas velocity from the mixer tank to the reaction zone.

The diameter of a mixing tank may be determined from the diameter of a corresponding reaction zone, the velocity for the reactant gas entering the reaction zone, and the superficial velocity of the gas in the mixing tank, assuming conservation of reactant gas volumetric flow between the mixer tank outlet and the reaction zone inlet. These variables are related by the equation:

4 Pi (Dr)² Vrg=4 Pi(Dt)² Vg

where:

Dt=tank diameter ; Dr=reaction zone diameter; Vrg=velocity of reactant gas entering the reaction zone (i.e. 3-6100 cm/sec); Vg=superficial velocity of gas in the mixing tank (i.e. 5-60 cm/sec); Pi=3.14

Therefore, Dt=Dr. square root (Vrg/Vg)

As the turbulence in the liquid increases, the collisions and coalescence/breaking events between bubbles are increased, leading to increased mixing. High liquid turbulence also tends to break-up larger bubbles and leads to a smaller average bubble size in the liquid. A preferred flow regime in the mixer tank is characterized by a churn-turbulent flow regime, wherein the total gas superficial velocity (corresponding to the combined gas flows) is between about 10 cm/sec and about 60 cm/sec. In the churn-flow regime, the gas-induced liquid turbulent flow should be sufficient to mix the plurality of gases. The use of mechanical devices, static structure, and/ore gas recirculation may be used, but should not be necessary. When the total gas superficial velocity is less than about 10 cm/sec, the flow in the mixer tank can be characterized by a bubbly flow regime. In the bubbly flow regime, the gas-induced turbulent flow may not be sufficient to mix efficiently the plurality of gases; therefore it might be necessary to increase the liquid turbulence in at least a portion of the liquid by using at least one mechanical device such as a powered device, at least one static structure, and/or a gas recirculation loop in order to increase the total gas flow (i.e. the total gas superficial velocity) entering the mixer tank to increase bubble interactions, hence gas mixing. When the total gas superficial velocity is greater than about 60 cm/sec, the flow in the mixer tank can be characterized by a slug flow regime. In the slug flow regime, the gas-induced turbulent flow may not be sufficient to mix the plurality of gases; therefore it might be necessary to increase the liquid turbulence in at least a portion of the liquid by using static structures, such as packing material, at least one baffle, at least one perforated plate, and the like to increase bubble interactions. The liquid turbulence can be characterized by a Reynolds number greater than 20, preferably greater than 200. Alternatively, the flow pattern of the gas phase in the bubble mixer tank can be described by the gas superficial velocity and the gas Peclet number, which has the form Pe^{G}=U_{G}L/D_{G}, where U_{G} is the superficial gas velocity, L is the gas-expanded liquid height in the tank, and D_{G} is the gas dispersion coefficient. The gas dispersion coefficient is a function of the superficial gas velocity, gas holdup, and the tank diameter. The gas flow is preferably characterized by a gas Peclet number greater than 0.1, preferably greater than about 1, still more preferably greater than about 5.

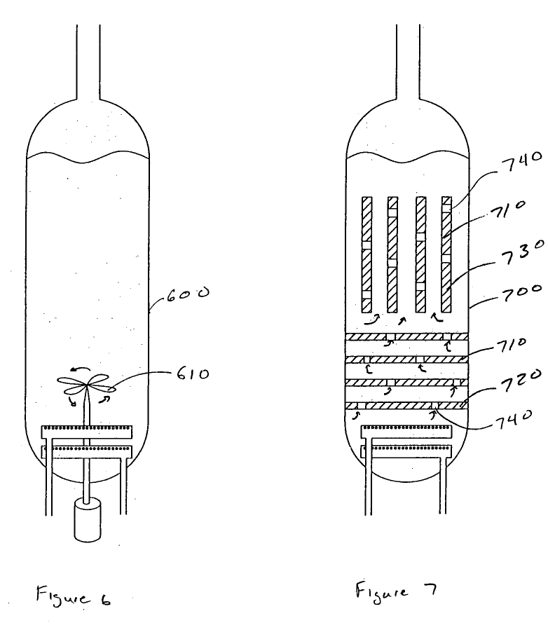

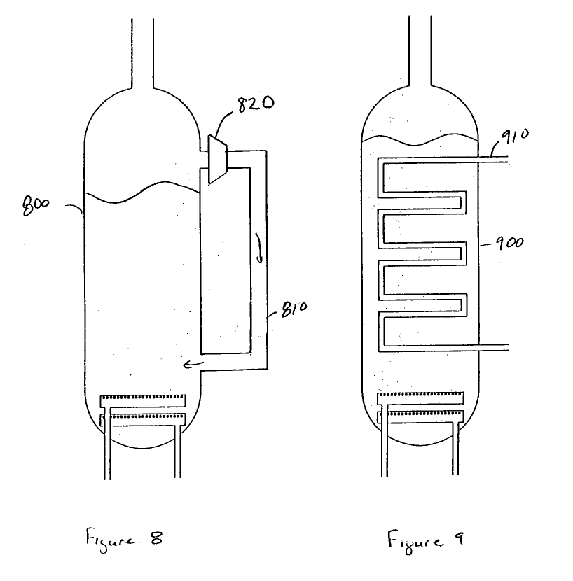

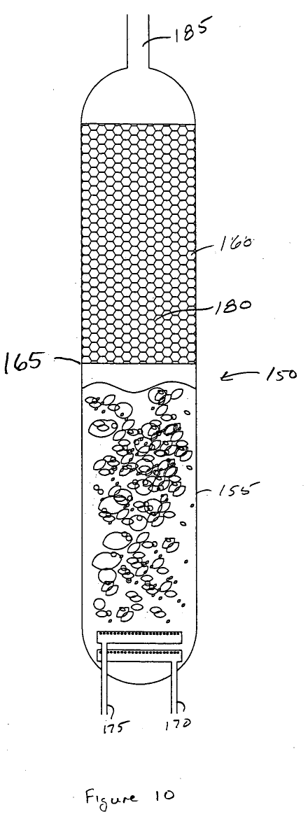

While some liquid turbulence is preferably induced by the injection of the feed gases, it may be desired to create additional turbulence within the liquid. The turbulence induced by gas flow may be supplemented by the help of a powered mechanical device, a recirculation loop for the gas, at least one static internal structure, or combinations thereof. For example, if the flow rate total gas superficial velocity is too low to cause a sufficiently turbulent liquid flow regime, then other additional means for creating the liquid turbulent flow may be needed. The powered mechanical device may comprise at least one paddle, at least one stirrer, at least one impeller, at least one propeller, or combinations thereof. The static internal structure may comprise at least one baffle, at least one partition plate, a packing material, a heat-exchange device, or combinations thereof. FIGS. 6-8 and 11 illustrate some possible techniques for creating additional turbulence within the liquid, namely mechanical stirrers (shown on FIG. 6), partition plates (shown on FIG. 7), fluid circulation systems, such as external gas recirculation with the use of a compressor (shown in FIG. 8) or an internal liquid recirculation with the use of a downcomer tube (not shown), and a packing material such as a random packing material (shown in FIG. 11) or a structured packing material (not illustrated).

FIG. 6 depicts one embodiment of a mixing tank 600 having mechanical stirrers 610 disposed in the fluid. Stirrers 610 agitate the fluid by rotating or oscillating. Stirrers 610 can be located on the bottom, sides, or top of tank 600 as desired. Multiple stirrers (not shown) can be used in order to create alternating breaking-up and coalescing zones within the liquid volume; the size of each zone would be mainly dependent on the agitation speed of the stirrers and the spacing between stirrers. FIG. 7 illustrates an alternative embodiment of a mixing tank 700 having partition plates 710 disposed in the fluid. Partition plates 710 may be oriented horizontally 720 or vertically 730 and may contain holes 740 to control the circulation of fluid through the plates. Partition plates 710, as well as other internal structures not shown in FIG. 7, such as baffles or packing material, hinder fluid flow and enhance the gas-induced liquid turbulence, as well as cause bubbles to coalesce and break. Packing material in mixing tank 700 may comprise random or structured packing material. As shown in FIG. 11, mixing tank 900 may contain packing material 920. Packing material 920 may be used to hinder fluid flow, enhance gas-induced liquid turbulence, and cause bubbles to coalesce and break. Packing material in mixing tank 900 may comprise random packing 920, such as rings, saddles, and/or balls, but may also comprise structured packing (not shown) such as trays, perforated plates, and corrugated sheet assemblies, such as those commercially available from Koch-Glitsch, LP or Jaeger Products, Inc.

Another alternate embodiment, including a mixing tank 800 having a fluid circulation system 810, is shown in FIG. 8. Fluid circulation system 810 draws gas from the upper portion of tank 800 and recycles the gas to the lower portion of the tank. System 810 preferably includes a compressor 820 such that the gas returned to tank 800 is at an elevated pressure. The elevated pressure helps to increase circulation and turbulence within tank 800. Although shown with a single compressor and inlet, alternate embodiments of fluid circulation systems 810 may include multiple inlets and outlets at various levels within a tank.

The above described means for increasing turbulence can be used solely or in any combination as needed to achieve sufficient turbulence to mix the desired reactant gas. It is also understood that the size and volume of the mixing tank contribute to the overall mixing potential of the system and therefore the mixing tank should be carefully selected for the gas volumes and flow rates that are being considered. Therefore, mixing tanks designed in accordance with the concepts of the present invention may be of any shape, size, or configuration and may or may not include means for increasing turbulence.

As previously discussed, for most oxidation reactions it is often desired to preheat the reactant gas to an elevated temperature from 25 to 300° C. before contacting the catalyst contained in a reactor. Therefore, as shown in FIG. 9, a preferred mixing tank 900 may include heating tubes 910 disposed within the liquid in the tank. A heating medium, such as saturated or superheated steam or hot oil or hot gas products, can be circulated through tubes 910 so as to transfer heat to the liquid and gas circulating in tank 900. Alternatively, the liquid in the tank may be heated in a heating unit outside of the tank and then circulated through the tank. Heating may also be by way of electrical resistance heating coils within the tank or any other practical method of heating. Any means for supplying heat to the reactant gas may also be combined with one or more means for increasing turbulence as discussed above.

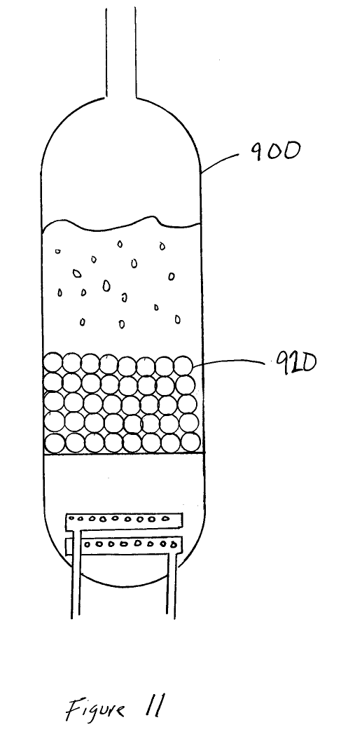

Referring now to FIG. 10, a catalytic oxidation system 150 includes a gas-induced liquid-turbulent mixing region 155 and a reaction region 160 comprising a catalyst 180, both regions being integrated into a single vessel 165. A hydrocarbon gas stream 170 and oxidant gas stream 175 are injected into the mixing region 155. The feed gas streams are mixed as they move through the liquid in mixing region 155 such the desired reactant gas exits the top of the gas-expanded mixing region 155 and enters reactor region 160. In reactor region 160 the reactant gases contact catalyst 180 and react to form product gases that exit vessel 165 through outlet 185. Mixing region 155 may include a means for increasing liquid turbulence for improving the mixing characteristics of the mixing region or means for supplying heat in order to deliver the reactant gas at the desired temperature. It should be understood that, even though reaction zone is shown comprising a catalyst 180, the oxidation system does not necessarily require a catalyst, and therefore a non-catalytic oxidation converting a reactant gas mixture without a catalyst is a suitable alternate embodiment of the oxidation system 150.

When the reactor shown in FIG. 2 or 10 is an oxidation reactor, the reactor can comprise any suitable reaction employing at least two feed gases.

In a particular preferred embodiment of the invention, when the oxidation reactor is used for the production of synthesis gas (syngas), the syngas reactor preferably employs the partial oxidation of a hydrocarbon-containing gas in the absence or presence of a catalyst. The hydrocarbon-containing feed is almost exclusively obtained as natural gas. However, the most important component in the hydrocarbon-containing feed is generally methane. Natural gas comprises at least 50% methane and as much as 10% or more ethane. Methane or other suitable hydrocarbon feedstocks (hydrocarbons with four carbons or less or C1-C4 hydrocarbons) are also readily available from a variety of other sources such as higher chain hydrocarbon liquids, coal, coke, hydrocarbon gases, etc., all of which are clearly known in the art. Preferably, the hydrocarbon feed comprises at least about 50% by volume methane, more preferably at least 80% by volume, and most preferably at least 90% by volume methane. The hydrocarbon feed can also comprise as much as 10% ethane. Similarly, the oxygen-containing gas may come from a variety of sources and will be somewhat dependent upon the nature of the oxidation reaction being used. For example, a partial oxidation reaction requires diatomic oxygen as the oxidant feedstock, while autothermal reforming reaction (another syngas production reaction) requires diatomic oxygen and steam as the oxidant feedstock. According to the preferred embodiment of the present invention, partial oxidation is assumed for at least part of the syngas production reaction.

The synthesis gas product contains primarily hydrogen and carbon monoxide, however, many other minor components may be present including steam, nitrogen, carbon dioxide, ammonia, hydrogen cyanide, etc., as well as unreacted feedstock, such as methane and/or oxygen. The synthesis gas product, i.e. syngas, is then ready to be used, treated, or directed to its intended purpose. The product gas mixture emerging from the syngas reactor may be routed directly into any of a variety of applications, preferably at pressure. For example, in the instant case some or all of the syngas can be used as a feedstock in subsequent synthesis processes, such as Fischer-Tropsch synthesis, alcohol (particularly methanol) synthesis, hydrogen production, hydroformylation, or any other use for syngas. One preferred such application for the CO and H₂ product stream is for producing in a synthesis reactor such as employing the Fischer-Tropsch reaction, higher molecular weight hydrocarbons, such as C₅₊ hydrocarbons. The syngas might need to be transitioned to be useable in a Fischer-Tropsch or other synthesis reactors. The syngas may be cooled, dehydrated (i.e., taken below 100° C. to knock out water) and compressed during the transition phase.

The synthesis reactor using syngas as feedstock is preferably a Fischer-Tropsch reactor. The Fischer-Tropsch reactor can comprise any of the Fischer-Tropsch technology and/or methods known in the art. The hydrogen to carbon monoxide ratio in the feedstock is generally deliberately adjusted to a desired ratio of between 1.4:1 to 2.3:1, preferably between 1.7:1 to 2.1:1, but can vary between 0.5 and 4. The syngas is then contacted with a Fischer-Tropsch catalyst in a Fischer-Tropsch reactor. The literature is replete with particular embodiments of Fischer-Tropsch reactors and Fischer-Tropsch catalyst compositions. Fischer-Tropsch catalysts are well known in the art and generally comprise a catalytically active metal, a promoter and a support structure. The most common catalytic metals are Group VIII metals from the Periodic Table of Elements, such as cobalt, nickel, ruthenium, and iron or mixtures thereof. The support is generally alumina, titania, zirconia, silica, or mixtures thereof. Fischer-Tropsch reactors use fixed and fluid type conventional catalyst beds as well as slurry bubble tanks. As the syngas feedstock contacts the catalyst, the hydrocarbon synthesis reaction takes place. The Fischer-Tropsch product contains a wide distribution of hydrocarbon products with a number of carbon atoms from C₅ to greater than C₁₀₀. The Fischer-Tropsch process is typically run in a continuous mode. In this mode, the gas hourly space velocity through the reaction zone typically may range from about 50 to about 10,000 hr⁻¹, preferably from about 300 hr⁻¹ to about 2,000 hr⁻¹. The gas hourly space velocity is defined as the volume of reactants at standard pressure and temperature per time per reaction zone volume. The reaction zone volume is defined by the portion of the reaction vessel volume where reaction takes place and which is occupied by a gaseous phase comprising reactants, products and/or inerts; a liquid phase comprising liquid/wax products and/or other liquids; and a solid phase comprising catalyst. The reaction zone temperature is typically in the range from about 160° C. to about 300° C. Preferably, the reaction zone is operated at conversion promoting conditions at temperatures from about 190° C. to about 260° C. The reaction zone pressure is typically in the range of about 80 psia (552 kPa) to about 1000 psia (6895 kPa), more preferably from 80 psia (552 kPa) to about 600 psia (4137 kPa), and still more preferably, from about 140 psia (965 kPa) to about 500 psia (3447 kPa).

The embodiments set forth herein are merely illustrative and do not limit the scope of the invention or the details therein. It will be appreciated that many other modifications and improvements to the disclosure herein may be made without departing from the scope of the invention or the inventive concepts herein disclosed. For example, although the Figures illustrate embodiments with one mixer and one reaction zone, it is envisioned that a multitude of such mixers could be used for one reaction zone. In addition, one mixer may supply a reactant gas mixture to a multitude of reaction zones, either placed in parallel or in series. Because many varying and different embodiments may be made within the scope of the inventive concept herein taught, including equivalent structures or materials hereafter thought of, and because many modifications may be made in the embodiments herein detailed in accordance with the descriptive requirements of the law, it is to be understood that the details herein are to be interpreted as illustrative and not in a limiting sense.

What is claimed is:

1. A reactor system comprising:
a tank comprising a bottom half and containing a liquid;
one hydrocarbon gas inlet located in the bottom half of the tank, wherein the hydrocarbon gas inlet comprises means of dispersing a hydrocarbon-containing gas into bubbles within said liquid;
one oxidant gas inlet located near or at the bottom of the tank, wherein the oxidant gas inlet comprises means of dispersing an oxygen-containing gas into bubbles within said liquid;
means of forming a gas-induced liquid turbulent region in at least a portion of said liquid sufficient to mix said bubbles of oxygen-containing gas and hydrocarbon-containing gas to provide a reactant gas; and
a reactor body in fluid contact with said tank adapted to receive the reactant gas at conditions favorable for the production of reaction products.

2. The system of claim 1 wherein the tank comprises a column with a height-to-diameter aspect ratio greater than 1 and not more than 15.

3. The system of claim 1 wherein the hydrocarbon-containing gas comprises methane.

4. The system of claim 1 wherein the oxygen-containing gas comprises molecular oxygen.

5. The system of claim 1 wherein the means of forming a gas-induced liquid turbulent region comprises a powered mechanical device, a fluid circulation system, a static internal structure, or combination thereof.

6. The system of claim 5 wherein the powered mechanical device comprises at least one paddle, at least one stirrer, at least one impeller, at least one propeller, or combinations thereof.

7. The system of claim 5 wherein the static internal structure comprises at least one baffle, at least one perforated plate, a packing material, a heat-exchange device, or combinations thereof.

8. The system of claim 1 wherein the means of forming the gas-induced liquid turbulent region employs passing a gas superficial velocity of the combined hydrocarbon-containing gas and oxygen-containing gas between about 5 and about 60 cm/sec through a portion of said liquid.

9. The system of claim 1 further comprising a means for heating or cooling.

10. The system of claim 1 wherein said tank and said reactor body are integrated into a single vessel.

12. The system of claim 1 wherein said reactor body comprises a partial oxidation reaction.

13. A method for forming a reactant gas mixture in a safe and efficient manner before being reacted, comprising the steps of:
providing a tank containing a liquid;
injecting a first feed gas into said liquid in a manner effective to subdivide the first feed gas into bubbles within the liquid;
separately injecting a second feed gas into said liquid in a manner effective to subdivide the second feed gas into bubbles within the liquid;
forming a gas-induced liquid turbulent region in at least a portion of said liquid;
passing bubbles of said first and second feed gases through said gas-induced liquid turbulent region so as to induce gas transfer between the bubbles and to form a reactant gas mixture comprising the first and second feed gases; and
supplying at least a portion of the reactant gas mixture to a reaction zone.

14. The method of claim 13 wherein forming a gas-induced liquid turbulent region employs passing a gas superficial velocity of the combined first and second gases between about 5 cm/sec and about 60 cm/sec.

15. The method of claim 14 wherein forming a gas-induced liquid turbulent region further includes using a powered mechanical device, a fluid circulation system, a static internal structure, or combination thereof.

16. The method of claim 13 wherein the first feed gas comprises a hydrocarbon gas and the second feed gas comprises an oxygen-containing gas.

17. The method of claim 16 wherein the reactant gas mixture has a O₂-to-carbon molar ratio between about 0.1:1 and about 0.8:1.

18. The method of claim 16 wherein the reactant gas mixture has a O₂-to-carbon molar ratio between about 0.45:1 and about 0.65:1.

19. The method of claim 13 further comprising maintaining a pressure between about 300 kPa-3350 kPa psig within the tank.

20. The method of claim 13 wherein the tank comprises a column with a height-to-diameter aspect ratio between 1 and 15.

21. The method of claim 20 further comprising heating the reactant gas mixture to a predetermined temperature before supplying the reactant gas mixture to the reactor.

22. The method of claim 20 wherein the liquid comprises water, an organic liquid, or combinations thereof.

23. A method for the oxidation of hydrocarbons comprising:
providing a tank containing a liquid;
injecting a hydrocarbon gas into said liquid in a manner effective to subdivide the hydrocarbon gas into bubbles within the liquid;
separately injecting an oxygen-containing gas into said liquid in a manner effective to subdivide the oxygen-containing gas into bubbles within the liquid;
forming a gas-induced liquid turbulent region in at least a portion of said liquid;
passing bubbles of the hydrocarbon gas and of the oxygen-containing gas through said gas-induced liquid turbulent region so as to induce gas transfer between the bubbles and to form a reactant gas mixture comprising the hydrocarbon gas and the oxygen-containing gas;
supplying at least a portion of the reactant gas mixture to a reactor, and
reacting at least a portion of said hydrocarbon gas with oxygen to form a reaction product.

24. The method of claim 23 wherein forming a gas-induced liquid turbulent region employs passing a gas superficial velocity of the combined hydrocarbon gas and oxygen-containing gas between about 5 cm/sec and about 60 cm/sec.

25. The system of claim 24 wherein the gas superficial velocity is between 10 and 45 cm/sec.

26. The method of claim 23 wherein forming a gas-induced liquid turbulent region include using a powered mechanical device, a fluid circulation system, a static internal structure, a high gas velocity, or combination thereof.

27. The method of claim 23 further comprising maintaining a pressure between about 300 kPa and about 3350 kPa psig within the tank.

28. The method of claim 23 wherein the tank comprises a column with a height-to-diameter aspect ratio between 1 and 15.

29. The method of claim 23 wherein the reactant gas mixture has a O₂-to-carbon molar ratio between about 0.1:1 and about 0.8:1.

30. The method of claim 29 wherein the reactant gas mixture has a O₂-to-carbon molar ratio between about 0.45:1 and about 0.65:1.

31. The method of claim 23 further comprising heating the reactant gas mixture to a predetermined temperature before supplying the reactant gas mixture to the reactor.

32. The method of claim 23 wherein the liquid comprises water, an organic liquid, or combinations thereof.

33. The method of claim 32 wherein the organic liquid comprise a hydrocarbon liquid or a mixture of liquid hydrocarbons.

34. The method of claim 23 wherein the reactor comprises a partial oxidation, and the reaction product comprises hydrogen and carbon monoxide.

35. The method of claim 34 wherein the partial oxidation comprises a catalyst.

36. The method of claim 23 wherein the reactant gas mixture further comprises at least a portion of said liquid.

37. A process for producing C₅₊ hydrocarbons comprising:
providing a tank containing a liquid;
injecting a hydrocarbon gas into said liquid in a manner effective to subdivide the hydrocarbon gas into bubbles within the liquid;
separately injecting an oxygen-containing gas into said liquid in a manner effective to subdivide the oxygen-containing gas into bubbles within the liquid;
forming a gas-induced liquid turbulent region in at least a portion of said liquid;
passing bubbles of the hydrocarbon gas and of the oxygen-containing gas through said gas-induced liquid turbulent region so as to induce gas transfer between the bubbles and to form a reactant gas mixture comprising the hydrocarbon gas and the oxygen-containing gas;
supplying at least a portion of the reactant gas mixture to a partial oxidation reactor;
reacting at least a portion of said hydrocarbon gas with oxygen in the a partial oxidation reactor to form a syngas stream comprising carbon monoxide and hydrogen;
feeding at least a portion of the syngas stream to a hydrocarbon synthesis reactor comprising a hydrocarbon synthesis catalyst; and
converting at least a portion of said syngas stream in the hydrocarbon synthesis reactor to form C₅₊ hydrocarbons.
